(19) United States
(12) Patent Application Publication
(10) Pub. No.: US 2013/0047887 A1
(43) Pub. Date: Feb. 28, 2013

(54) NOVEL MULTIFUNCTIONAL MOLECULES FOR DENTAL BONDING APPLICATIONS HAVING IMPROVED ADHESION

(71) Applicants: Marianela Trujillo-Lemon, Louisville, CO (US); Kristina L. Esquibel, Louisville, CO (US); Amy J. Docktor, Louisville, CO (US); Zachary R. Shelton, Louisville, CO (US); Jeffrey M. Leadford, Louisville, CO (US); Kathryn T. Ida, Louisville, CO (US); Cora Bracho-Troconis, Louisville, CO (US)

(72) Inventors: Marianela Trujillo-Lemon, Louisville, CO (US); Kristina L. Esquibel, Louisville, CO (US); Amy J. Docktor, Louisville, CO (US); Zachary R. Shelton, Louisville, CO (US); Jeffrey M. Leadford, Louisville, CO (US); Kathryn T. Ida, Louisville, CO (US); Cora Bracho-Troconis, Louisville, CO (US)

(73) Assignee: SEPTODONT, CONFI-DENTAL DIVISION, Louisville, CO (US)

(21) Appl. No.: 13/499,894

(22) PCT Filed: Oct. 1, 2010
(86) PCT No.: PCT/US10/51122

§ 371 Date: Oct. 24, 2012

Related U.S. Application Data
(60) Provisional application No. 61/247,797, filed on Oct. 1, 2009.

(51) Int. Cl. A61K 6/00; C07D 309/30; C07F 9/40; C07C 69/767; C07C 69/608

(52) U.S. Cl. 106/35; 560/85; 560/127; 558/180; 558/172; 549/292

(57) ABSTRACT

The present invention describes dental adhesive compositions used for bonding dental biomaterials to hard tissue comprising a polymerizable blend of one or more newly synthesized low shrinkage, stable, multifunctional compounds, where the compounds are acidic-methacrylate derivatives, having excellent properties of bonding the hard tooth substance (enamel or dentin) to dental restorative materials, and present high quality marginal sealing between the tooth and the material thus bond and improved storage stability

### FIELD OF THE INVENTION

The invention relates to multifunctional molecules containing acidic polymerizable monomers, processes for making the monomers and compositions comprising the monomers. The invention relates to primer compositions and to adhesive bonding systems using such compositions. The present invention also comprises methods of using the monomers, such as in dental applications, and in particular, dental total-etching or self-etching adhesives in a single-component or in a multi-component presentation.

### BACKGROUND OF THE INVENTION

Dental adhesives have dramatically changed the options available for restoration placements since their introduction more than forty years ago. Initially, the use of dental adhesives required a long etching time and were only recommended for etching and bonding of the enamel. Dental bonding systems have evolved and become more effective with advances in chemistry, application, mechanism, and technique.

Currently used dental adhesives contain different monomer components in addition to the initiator system and solvent, such as water, ethanol, acetone or mixtures thereof. The requirements for enamel-dentin adhesives include removal of the smear layer on top of the dentin, creation of an adequate etch pattern (demineralization) on the tooth structure in a short period of time, and diffusion of monomers into etched enamel and dentin.

For the purpose of the present invention, dental adhesives will be classified into two main groups, etch and rinse (“total etch”) and a self-etching type. In the case of total etch adhesives, the surface of the tooth is treated or etched beforehand with a solution of phosphoric acid, and then, while the tooth is still wet from water cleansing, the adhesive is applied with a bonding agent. In the case of self-etching adhesives, the tooth surface is thoroughly dried by application of blowing air and subjected, without any pretreatment, to application of an acidic-bonding agent having a function of an etching agent. A bonding layer can be applied simultaneously to the surface of the tooth. By the use of the self-etching bonding agents, the pretreatment process with phosphoric acid is eliminated.

Ideally, the dental professional would use the etching, priming and adhesive bonding system in a manner that minimizes the time required for a patient to be in the dental chair. In addition, an ideal etching, priming and adhesive bonding system would also provide consistently high and stable bond strength of adhesives, composites, resins, metals and other dental prostheses to dentin and enamel. In the field of dental adhesives, several materials have been developed having excellent adhesiveness to a tooth, especially to the enamel portion. However, there is a need in the art for dental adhesives that provide further improved adhesion as well as no marginal gap formation when a dental restorative material is applied to a tooth.

### DESCRIPTION OF THE RELATED ART

Yeniad et al., “Synthesis and photopolymerization of new phosphonated monomers for dental applications,” Journal of Polymer Science: Part A: Polymer Chemistry, Vol. 46, No 6 (2008): pp. 2290-2299, discloses the synthesis of phosphonate monomers from the reaction of glycidyl methacrylate with (diethoxy-phosphoryl)acetic acid or (2-hydroxy-ethyl)-phosphonic acid dimethyl ester.

Youssef et al. “New phosphonated methacrylates: Synthesis, photocuring and study of their thermal and flame-retardant properties,” Macromol. Chem. Phys., Vol. 204 (2003), 1842-1850, discloses the synthesis of methacrylate phosphonate monomer according to two different pathways.

Brunet et al., “Solid-state reshaping on nanostructured crystals: supramolecular chirality of layered materials derived from polyethylenoxa-pillared zirconium phosphate,” Tetrahedron: Asymmetry, vol. 17 (2006): pp. 347-354, discloses the synthesis of diethyl[2-(oxyran-2-ylmethoxy)ethyl]phosphonate from the reaction of diethyl-vinylphosphonate and glycidol in the presence of CsCO₃ and its reaction with hexaethyleneglycol.

U.S. Patent Application 2009/0043008 A1 discloses a one part self-etching, self-priming dental adhesive with improved storage stability due to use of a specific thermal polymerization inhibitor, self priming dental adhesive having pH of at most 2.

U.S. Patent Application 2008/0194730A1 discloses an aqueous one-pack self-etching and self-priming dental adhesive composition having a pH of at most 2, which comprises: (i) a polymerizable N-substituted alkylacrylic or acrylic amide monomer with an optional inorganic acidic moiety selected from a phosphonic acid or sulfonic acid, and (ii) a curing system.

U.S. Pat. No. 4,612,384 shows a polymerizable composition containing phosphate monoester adhesive compositions.

### SUMMARY OF THE INVENTION

There is an unmet need for compounds that can be used in dental adhesive compositions that have excellent bonding properties, high quality marginal sealing, and improved storage stability. The present invention addresses the unmet needs in the art and provides novel low shrinkage multifunctional molecules for dental bonding applications, having improved adhesion and which exhibit improved properties of bonding the hard tooth substance (i.e. enamel, dentin) to dental restorative materials. The molecules also form a high quality seal between the tooth and the material bonded thereto and provide improved storage stability.

The present invention provides for compounds which are acidic-methacylate derivatives, and compositions comprising such compounds.

The present invention also provides for methods for producing the acidic-methacylate derivatives.

The present invention also provides for methods of using compositions comprising the acidic-methacrylic derivatives in dental applications.

### DETAILED DESCRIPTION OF THE INVENTION

The present invention relates to a compound selected from the group consisting of:

(I) a compound of formula (I):

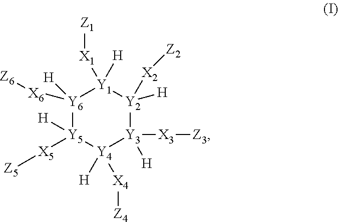

wherein the ring structure of formula (I) is preferably saturated or contains up to three unsaturations, and wherein:
   Y₁-Y₆, each independent from each other, is selected from the group consisting of: C, O, N, and S, with the proviso that at least three of Y₁-Y₆ are C, and wherein:
   (i) when any one of Y₁-Y₆ is O, S, or an unsaturated nitrogen then the corresponding H, X₁-X₆ and Z₁-Z₆ are absent;
   (ii) when any one of Y₁-Y₆ is a saturated nitrogen or an unsaturated carbon, then the corresponding H is absent;
   X₁-X₆, each independent from each other, is a direct bond, or is selected from the group consisting of: ═O, ═S, ═N—, and R_{X}, wherein when any one of X₁-X₆ is ═O or ═S, then the corresponding Z₁-Z₆ is absent, wherein R_{X} is a C₁-C₁₅ group optionally having at least one unsaturation, branch and/or cycle, which is substituted up to 4 times or unsubstituted, and which may be interrupted by at least one O or S, wherein the substituents are each independently selected from the group consisting of —OH, —OR_{V}, ═O, ═S, —O₂CR_{V}, —SH, —SR_{V}, —SOCR_{V}, —NH₂, —NHR_{V}, —N(R_{V})₂, —NHCOR_{V}, —NRCOR_{V}, —I, —Br, —Cl, —F, —CN, —CO₂H, —CO₂R_{V}, —CHO, —COR_{V}, —CONH₂, —CONHR_{V}, —CON(R_{V})₂, —COSH, —COSR_{V}, —NO₂, —SO₃H, —SOR_{V}, and —SO₂R_{V}, wherein R_{V} is a linear, branched or cyclic alkyl of one to ten carbon atoms,
   Z₁-Z₆, each independent from each other, is R_{S}, wherein R_{S} is selected from the group consisting of:
      (a) H;
      (b) a radical of formula (IV)

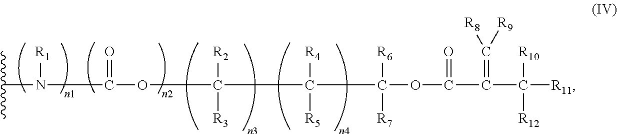

wherein:
   n1 and n2, each independent from each other, is 0 or 1;
   n3 and n4, each independent from each other, is 0 to 6;
   R₁ to R₁₂, each independent from each other, is selected from the group consisting of:
      (i) H,
      (ii) R_{Y}, wherein R_{Y} is C₁-C₆ group optionally having at least one unsaturation, branch and/or cycle, which is substituted up to 2 times or unsubstituted, and which may be interrupted by at least one O or S, wherein the substituents are each independently selected from the group consisting of —OH, —OR, ═O, ═S, —O₂OR, —SH, —SR, —SOCR, —NH₂, —NHR, —N(R)₂, —NHCOR, —NRCOR, —I, —Br, —Cl, —F, —ON, —CO₂H, —CO₂R, —CHO, —COR, —CONH₂, —CONHR, —CON(R)₂, —COSH, —COSR, —NO₂, —SO₃H, —SOR, and —SO₂R, wherein R is a linear or branched alkyl of one to three carbon atoms, and
      (iii) a radical of formula (V):

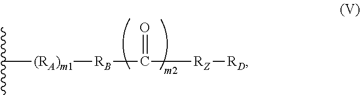

wherein:
   R_{A} is R_{X}, as defined above, and m1 is 0 or 1,
   R_{B} is O or S,
   m2 is 0 or 1;
   R_{Z} is selected from the group consisting of:

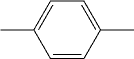

and a C₁-C₃ group optionally having at least one unsaturation or branch, which is substituted up to 2 times or unsubstituted, and which may be interrupted by at least one O or S, wherein the substituents are each independently selected from the group consisting of —OH, —OR, ═O, ═S, —O₂CR, —SH, —SR, —SOCR, —NH₂, —NHR, —N(R)₂, —NHCOR, —NRCOR, —I, —Br, —Cl, —F, —CN, —CO₂H, —CO₂R, —CHO, —COR, —CONH₂, —CONHR, —CON(R)₂, —COSH, —COSR, —NO₂, —SO₃H, —SOR, and —SO₂R, wherein R is a linear or branched alkyl of one to three carbon atoms,
R_{D} is selected from the group consisting of:
(i)

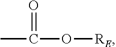

wherein R_{E} is H or R_{Y}, as defined above; and
(ii)

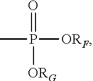

wherein R_{F} and R_{G}, each independent from each other, are selected from the group consisting of: H and R_{Y}, as defined above; and wherein at least one of R₁ to R₁₂ is a radical of formula (V); and

(II) a compound of formula (II):

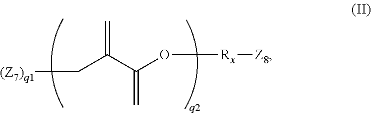

wherein:
   Z₇ is R_{S}, as defined above,
   q1 and q2, each independent from each other, is 0 or 1, and preferably, when q2 is 1, then q1 is 0
   R_{X} is as defined above, and
   Z₈ is a radical of formula (IV), as defined above; and

(III) a compound of formula (III):

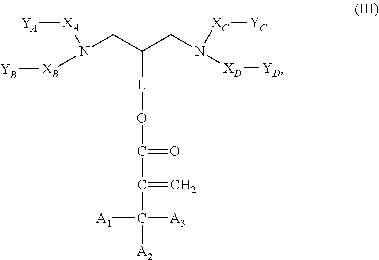

wherein:
   X_{A}, X_{B}, X_{C}, and X_{D}, each independent from each other, is a direct bond or R_{Y}, wherein R_{Y} is C₁-C₆ group optionally having at least one unsaturation, branch and/or cycle, which is substituted up to 2 times or unsubstituted, and which may be interrupted by at least one O or S, wherein the substituents are each independently selected from the group consisting of —OH, —OR, ═O, ═S, —O₂CR, —SH, —SR, —SOCR, —NH₂, —NHR, —N(R)₂, —NHCOR, —NRCOR, —I, —Br, —Cl, —F, —CN, —CO₂H, —CO₂R, —CHO, —COR, —CONH₂, —CONHR, —CON(R)₂, —COSH, —COSR, —NO₂, —SO₃H, —SOR, and —SO₂R, wherein R is a linear or branched alkyl of one to three carbon atoms, and preferably, X_{A}, X_{B}, X_{C}, and X_{D} are each unsubstituted C₁ alkyl groups;
   Y_{A}, Y_{B}, Y_{C}, and Y_{D}, each independent from each other, is H or COOH, with the proviso that at least two of Y_{A}, Y_{B}, Y_{C}, and Y_{D} are COOH, and preferably Y_{A}, Y_{B}, Y_{C}, and Y_{D} are each COOH;
   L is selected from the group consisting of:
   (a) a direct bond,
   (b)

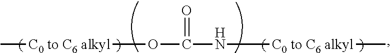

and
(c) R_{Y}, as defined above; and
A₁, A₂, and A₃, each independent of each other, are H or R_{Y} as defined above, and preferably A₁, A₂, and A₃ are each H.

In some preferred embodiments, in formula (I): (1) Y₁-Y₆ are all C; or (2) one of Y₁-Y₆ is O, and the remainder of Y₁-Y₆ are C. In some preferred embodiments, in formula (I), two of X₁-X₆ are ═O, and one of X₁-X₆ is ═N—. In some preferred embodiments, in formula (I), at least 3 of X₁-X₆ are R_{X}, wherein R_{X} is a C₅-C₉ alkyl.

In some preferred embodiments, in formula (IV), R₁-R₁₂ are H. In some preferred embodiments, in formula (IV), n3 and n4 are 1. In some preferred embodiments, in formula (IV), n2 is 1. In some preferred embodiments, in formula (IV), R₁ is H, n1 is 1, and n2 is 1. In some preferred embodiments, in formula (IV), R₄ or R₆ is a radical of formula (V).

In some preferred embodiments, in formula (V), R_{D} is

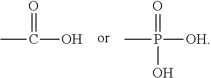

In some preferred embodiments, R_{Z} is a C₁-C₂ alkyl or

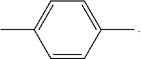

In some preferred embodiments, in formula (V), m2 is 1 and R_{B} is O. In some preferred embodiments, in formula (V), R_{A} is a C₁ alkyl and m1 is 1.

In some preferred embodiments, in formula (III), L is a direct bond or is selected from the group consisting of:

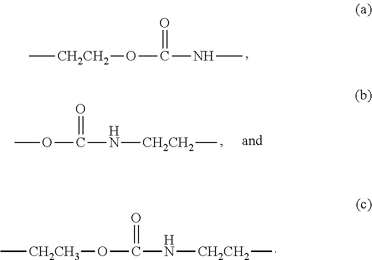

The present invention also relates to the compound of formula (1):

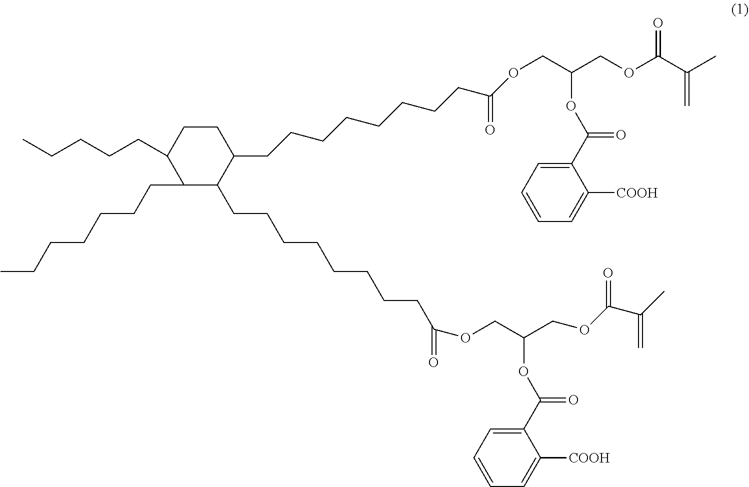 [SMILES: C=C(C)C(=O)OCC(COC(=O)CCCCCCCCC1CCC(CCCCC)C(CCCCCCC)C1CCCCCCCCC(=O)OCC(COC(=O)C(=C)C)OC(=O)c1ccccc1C(=O)O)OC(=O)c1ccccc1C(=O)O]

The present invention also relates to the compound of formula (2):

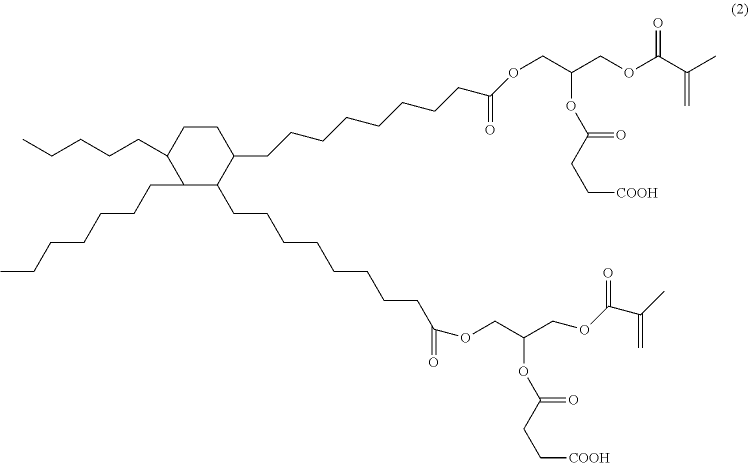 [SMILES: C=C(C)C(=O)OCC(COC(=O)CCCCCCCCC1CCC(CCCCC)C(CCCCCCC)C1CCCCCCCCC(=O)OCC(COC(=O)C(=C)C)OC(=O)CCC(=O)O)OC(=O)CCC(=O)O]

The present invention also relates to the compound of formula (3):

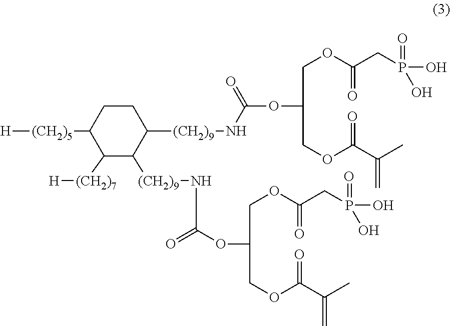

The present invention also relates to the compound of formula (4):

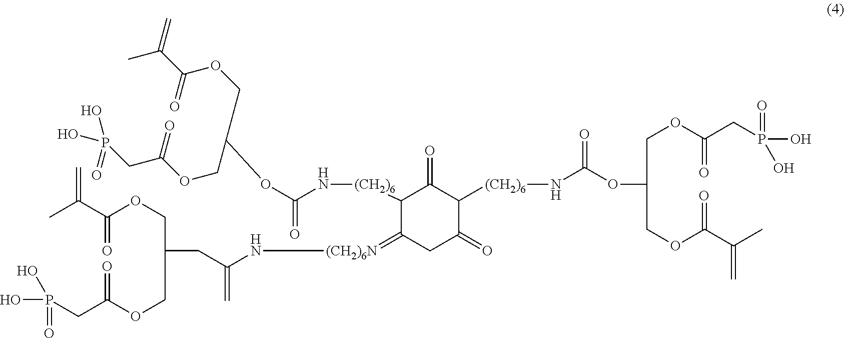

The present invention also relates to the compound of formula (5):

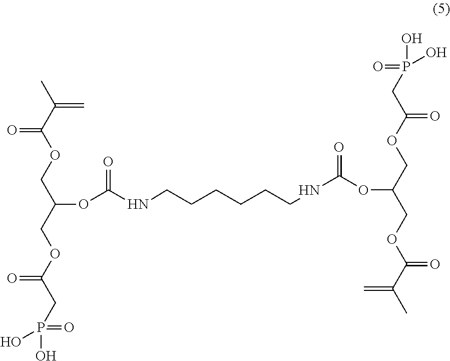 [SMILES: C=C(C)C(=O)OCC(COC(=O)CP(=O)(O)O)OC(=O)NCCCCCCNC(=O)OC(COC(=O)CP(=O)(O)O)COC(=O)C(=C)C]

The present invention also relates to the compound of formula (6):

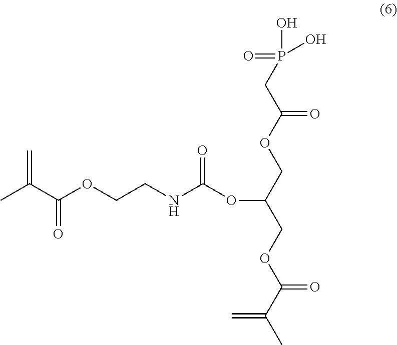 [SMILES: C=C(C)C(=O)OCCNC(=O)OC(COC(=O)CP(=O)(O)O)COC(=O)C(=C)C]

The present invention also relates to the compound of formula (7):

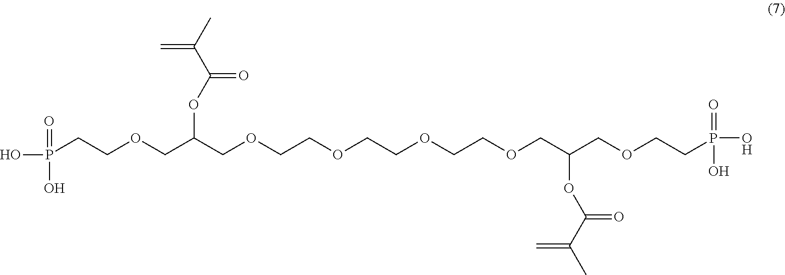 [SMILES: C=C(C)C(=O)OC(COCCOCCOCCOCC(COCCP(=O)(O)O)OC(=O)C(=C)C)COCCP(=O)(O)O]

The present invention also relates to the compound of formula (8)

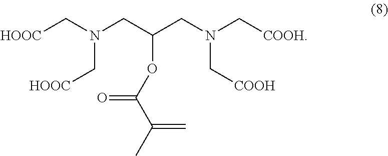 [SMILES: C=C(C)C(=O)OC(CN(CC(=O)O)CC(=O)O)CN(CC(=O)O)CC(=O)O]

The present invention also relates to the compound of formula (9)

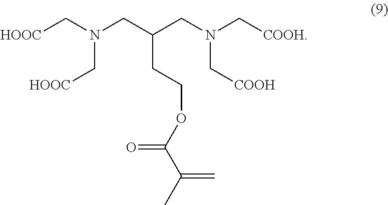 [SMILES: C=C(C)C(=O)OCCC(CN(CC(=O)O)CC(=O)O)CN(CC(=O)O)CC(=O)O]

The present invention also relates to the compound of formula (10)

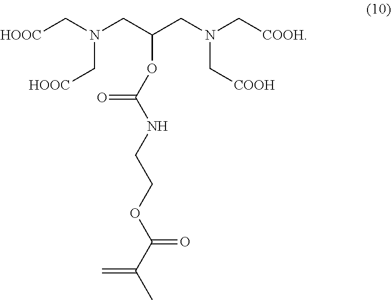 [SMILES: C=C(C)C(=O)OCCNC(=O)OC(CN(CC(=O)O)CC(=O)O)CN(CC(=O)O)CC(=O)O]

The present invention also relates to the compound of formula (11)

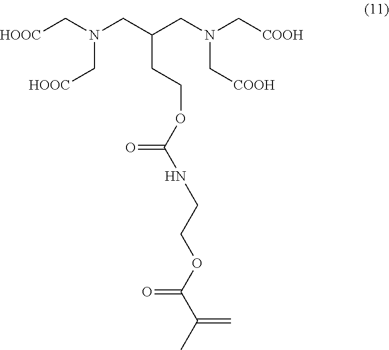 [SMILES: C=C(C)C(=O)OCCNC(=O)OCCC(CN(CC(=O)O)CC(=O)O)CN(CC(=O)O)CC(=O)O]

The present invention also relates to processes for producing the compounds of formula (I), (II), and (III).

The present invention also relates to a process for producing the compound of formula (1), comprising:

a) mixing diglycidyl ester, methacrylic acid, 4-dimethoxyphenol (BHT), and a base, wherein the base is preferably 4-dimethylaminopyridine (DMAP), triethylamine, or triphenyl phosphine,

b) adding phatallic anhydride dissolved in a polar solvent, wherein the solvent is preferably tetrahydrofuran.

The present invention also relates to a process for producing the compound of formula (2), comprising:

a) mixing diglycidyl ester, methacrylic acid, 4-dimethoxyphenol (BHT), and a base, wherein the base is preferably 4-dimethylaminopy dine (DMAP), triethylamine, triphenyl phosphine, or dimethylamino pyridine;

b) adding succinic anhydride dissolved in a polar solvent, wherein the solvent is preferably tetrahydrofuran.

The present invention also relates to a process for producing the compound of formula (3), comprising:

a) mixing 3-(2-diethoxyphosphoryl)acetoxy)-2-hydroxypropyl methacrylate with diisocyanate, or mixing 3-(2-diethoxyphosphoryl)acetoxy)-2-hydroxypropyl methacrylate with a carboxylic acid and reacting with a dehydrating agent such as N,N′-dicyclohexylcarbodiimide (DCC);

b) adding a catalyst preferably selected from the group consisting of: dibutyl tin dilaurate, KKAT A209 (a zirconium chelate complex dissolved in a reactive diluent and t-butyl acetate), zirconium acetylacetonate, and dioctyltin dilaurate (DOTDL);

c) dissolving the resulting mixture in methylene chloride and trimethylsilylbromide;

d) removing solvent; and

e) adding methanol.

The present invention also relates to a process for producing the compound of formula (4), comprising:
a) mixing 3-(2-diethoxyphosphoryl)acetoxy)-2-hydroxypropyl methacrylate with Desmodur XP2410

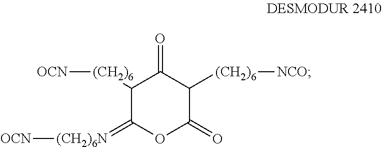

b) adding a catalyst preferably selected from the group consisting of: dibutyl tin dilaurate, KKAT A209 (a zirconium chelate complex dissolved in a reactive diluent and t-butyl acetate), zirconium acetylacetonate, and dioctyltin dilaurate (DOTDL);
c) dissolving the resulting mixture in methylene chloride and methylsilylbromide;
d) removing solvent; and
e) adding methanol.

The present invention also relates to a process for producing the compound of formula (5), comprising:

a) mixing 3-(2-diethoxyphosphoryl)acetoxy)-2-hydroxypropyl methacrylate with 1,6-hexanediisocyanate,
b) adding a catalyst preferably selected from the group consisting of: dibutyl tin dilaurate, KKAT A209 (a zirconium chelate complex dissolved in a reactive diluent and t-butyl acetate; King Industries, Norwalk, Conn.), zirconium acetylacetonate, and dioctyltin dilaurate (DOTDL);
c) dissolving the resulting mixture in methylene chloride and trimethylsilylbromide;
d) removing solvent; and
e) adding methanol.

The present invention also relates to a process for producing the compound of formula (6), comprising:

a) mixing 3-(2-diethoxyphosphoryl)acetoxy)-2-hydroxypropyl methacrylate with 2-isocyanoethyl methacrylate
b) adding a catalyst preferably selected from the group consisting of: dibutyl tin dilaurate, KKAT A209 (a zirconium chelate complex dissolved in a reactive diluent and t-butyl acetate; King Industries, Norwalk, Conn.), zirconium acetylacetonate, and dioctyltin dilaurate (DOTDL);
c) dissolving the resulting mixture in methylene chloride and trimethylsilylbromide;
d) removing solvent; and
e) adding methanol.

The present invention also relates to processes for producing the compound of formula (7), (8), (9), (10), and (11), comprising: protection of acid groups (ester formation), reaction of the alcohol with an acyl chloride (for example, methacryloyl chloride) in the presence of a base (for example, triethylamine), or reaction of the alcohol with an isocyanate (for example, isocyanatoethyl methacrylate) using a catalyst (for example Dibutyltin Dilaurate, or DBTDL), and cleavage of the protecting group.

The present invention also relates to a composition comprising a compound of formula (I), formula (II), or formula (III), or a compound of formulas (1)-(11). In some embodiments, the composition further comprises one or more polymerizable methacrylic monomers selected from the group consisting of: 2,2-bis[4-2(hydroxyl-3-methacryloyloxypropyl)phenyl]propane (Bis-GMA), dimer dicarbamate dimethacrylate (DDCDMA), 1,6-bis-[2-methacryloyloxyethoxycarbonylamino]-2,4,4-trimethylhexane (UDMA), and 2,2-bis(4-(2-Methacryloxyethoxy)phenylpropane (Bis-EMA), and Poly (ethylene glycol) dimethacrylate (PEGDMA).

In some embodiments, the composition further comprises hydrophilic methacrylate compounds selected from the group consisting of: 2-hydroxyethyl methacrylate (HEMA), triethylene glycol dimethacrylate (TEGDMA), ethylene glycoldimethacrylate (EGDMA), glycerol dimethcarylate (GDMA).

In some embodiments, the composition further comprises a water soluble organic solvent selected from the group consisting of alcohol or ketones including but not limited to ethanol, propanol, acetone, and methylethyl ketone.

In some embodiments, the compositions further comprise one or more filler materials or compounds. The composition may contain any filler material suitable for use in dental applications, including, but not limited to, silanized inorganic compounds. Filler materials include, but are not limited to, compounds which can increase viscosity and increase strength. In preferred embodiments, the compositions can comprise filler materials selected from the group consisting of: silanized inorganic compounds, silica, silicate glass, quartz, barium silicate, strontium silicate, barium borosilicate, strontium borosilicate, borosilicate, alumina, zirconia, tin oxide, ytterbium fluoride, and pigments.

In some embodiments, the compositions can comprise pigments or coloring agents, inhibitors, and/or initiator systems. In some embodiments, the particle sizes of the one or more filler materials are between about 0.001 to about 5.0 micrometers.

The present invention provides methods of using the compounds of formula (I), formula (II), or formula (III), or compounds (1)-(11) in dental applications. For example, the compounds may be used for dental applications including, but not limited to, dental adhesives; self adhesive restorative materials; permanent and temporary dental resin cements; light cure and chemical cure dental nanohybrid, microhybrid, and hybrid composites; dental nanohybrid and microhybrid flowable composites; temporary filling material; core build up material; and pit and fissure sealants.

In some embodiments, the compounds can be used in dental adhesives in bonding dental biomaterials to hard tissues via a separate acid etching (total etch) or through a self-etching step without preparation of the hard tissue substrate.

The compositions can be modified to affect properties such as pH, viscosity, rate of polymerization, final conversion, film thickness and bond strength. Thus, the adhesive compositions of the present invention may contain also contain in addition to the newly developed acid monomers: (a) one or more hydrophilic monomers in the amount of 5 to 90 wt %, preferably in the amount from 15 to 70 wt %, (b) one or more hydrophobic cross-linking compounds in the amount of 5 to 90 wt %, preferably in the amount from 20 to 70 wt %, (c) an organic water soluble solvent selected from the group of alcohols and ketones such as ethanol, propanol, acetone, methyl ethyl ketone; and (d) may or may not include water to hydrolyze the acid monomer and wet the hard tooth structure.

In some embodiments, the adhesive composition can also be used with at least one initiator to allow photo and/or chemical curing. In some embodiments, the composition may additionally contain a co-initiator to accelerate the curing process. A photopolymerization inhibitor may also be included in the adhesive composition in order to increase shelf life and/or stability.

The examples describe hereinafter are given for illustrative purpose only and are not intended to limit the scope of the invention.

### DESCRIPTION OF THE FIGURES

FIG. 1 shows the structure of 3-(2-(diethoxyphosphoryl)acetoxy)-2-hydroxypropyl methacrylate, which can be used as a starting material in the synthesis of compounds of the present invention.

FIG. 2 shows the structures of commercial materials which can be used in the synthesis of compounds of the present invention.

FIG. 3 shows the structure of polymerizable carboxylic acid compounds which can be used in compositions comprising the compounds of the present invention.

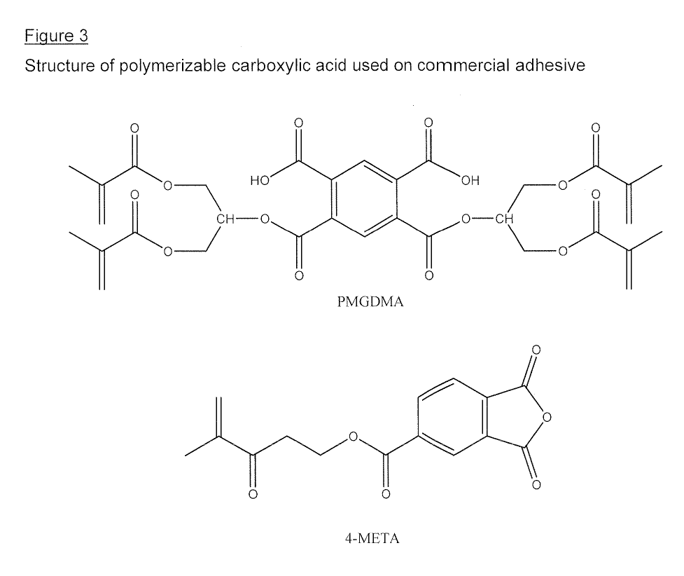

FIG. 4 shows the structures of commercially available monomers used in dental adhesive formulations.

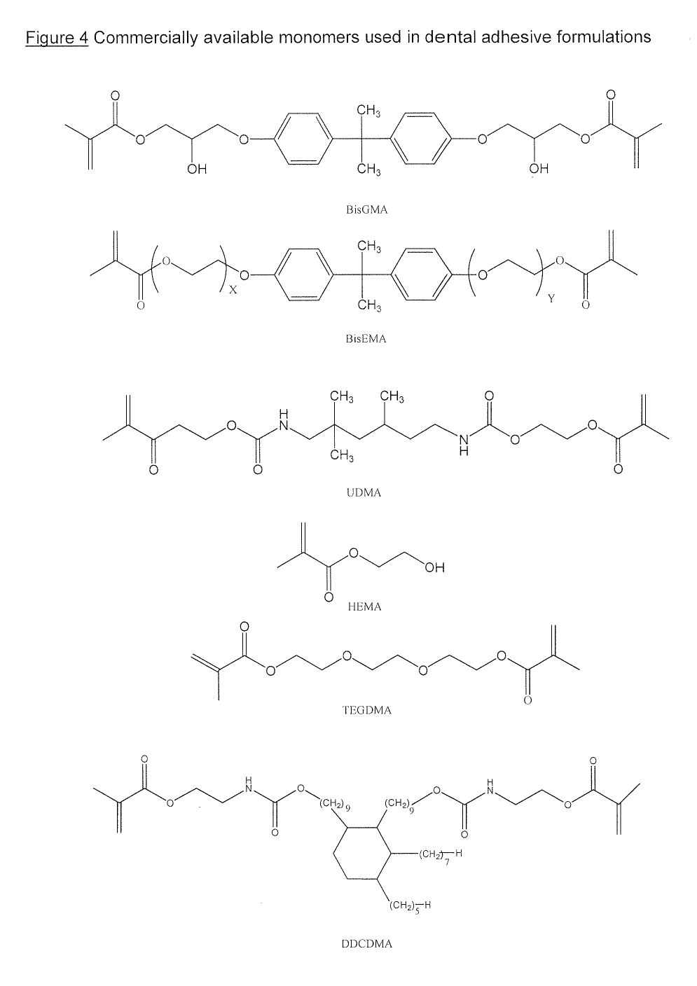

FIG. 5 shows pH values of Part A self-etching adhesive compositions compared with commercial products, as described in Example 1.

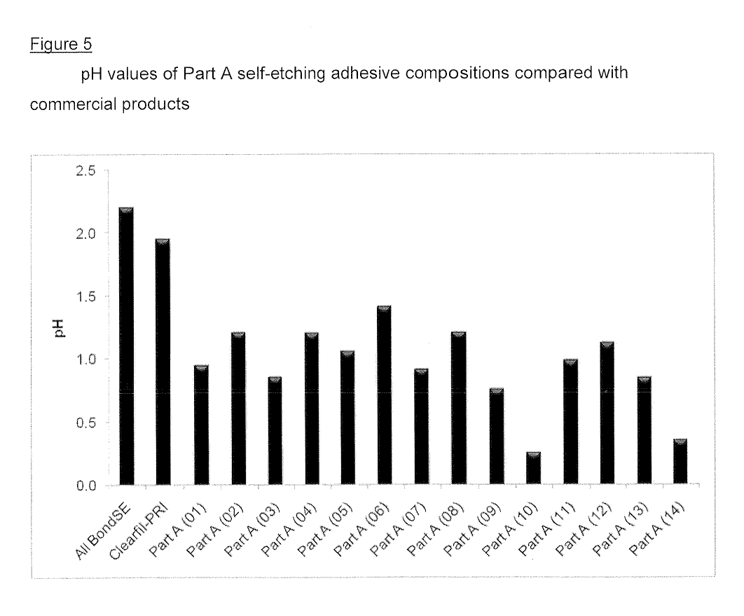

FIG. 6 shows water sorption and solubility for Part B, self-etching compositions, as described in Example 1.

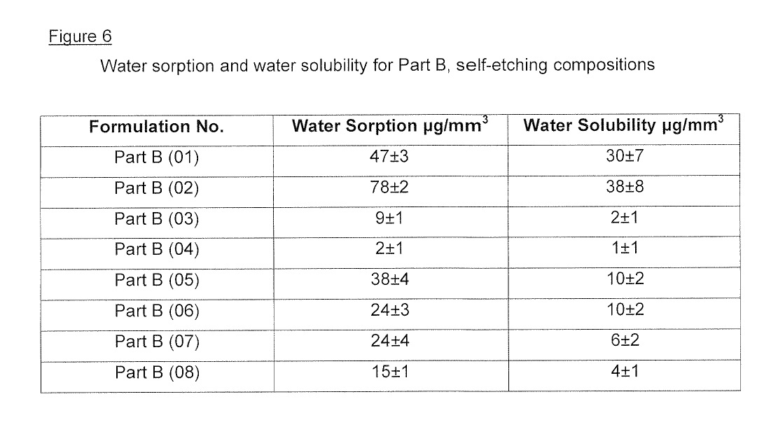

FIG. 7 shows double bond conversion values for Part B, self-etching compositions, as described in Example 1.

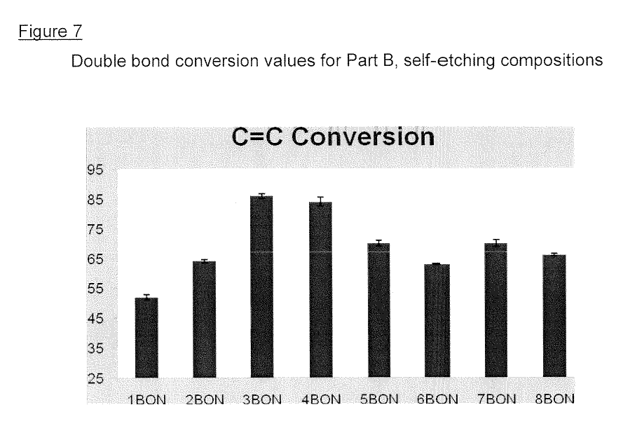

FIG. 8 shows flexural strength and Young's modulus for Part B, self-etching compositions, as described in Example 1.

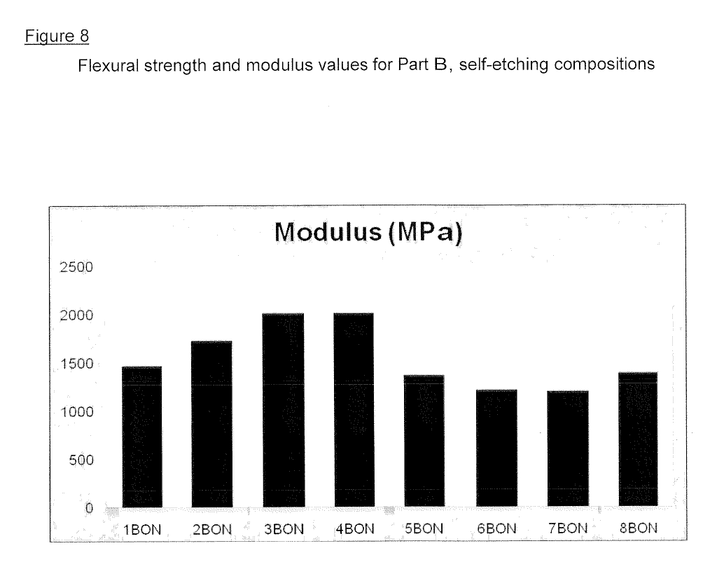

### EXAMPLES

### Example 1

### Materials, Methods and Instruments

The reactants and organic solvents utilized in the synthesis of the new monomers as well as known monomers and common resins were commercially obtained and were used as received. FT infrared spectra of thin films between KBr crystals were recorded on a Nicolet Nexus 670 spectrometer. The ¹H NMR and decouple ¹³C NMR spectra were obtained on a Varian Inova 500-MHz spectrometer using CDCl₃ as the solvent. Monomers viscosities were measured on pure monomers at 25° C. with a parallel-plate viscometer (CAP 2000+; Brookfield Engineering Laboratories, Stoughton, Mass.). The test was run with spindles CAP-S-01 (900 rpm) or CAP-S-06 (200 rpm) depending on resin viscosity for 15 seconds.

To permit photo-polymerization with visible light activation, 0.4 wt % camphorquinone and 0.8 wt % ethyl 4-N,N-dimethylaminobenzoate were mixed with the monomers, in some cases an acyl-phosphinoxide type photoinitiator was used. Dynamic and static photopolymerization studies were conducted with a visible light curing unit (Maxima Cure Power) on specimens prepared with a Delrin ring (inner dimensions: 1.25 mm thick and 12.5 mm diameter) sandwiched between glass cover slips irradiated for 40 s at 375 mW/cm². Dynamic and static measurements of the methacrylate monomers conversion were accomplished with transmission near-infrared (NIR) spectroscopy (Nexus 670, Nicolet). The conversion values were determined from the change in the peak area of the methacrylate overtone absorption (═C—H at 6165 cm⁻¹) before and after polymerization. Triplicate specimens of each monomer were polymerized and analyzed. Post-gel polymerization volumetric shrinkage was measured using an ACTA (Academic Center for Dentistry Amsterdam, Department of Materials Science, Amsterdam, The Netherlands) linometer. Polymer flexural strength and modulus were calculated using a three-point-bending test, carried out with a hydraulic universal test system (Instron, Norwood, Mass.). Water sorption and solubility were determinate according to ISO 4049. Shear bond strength test was carried out according to procedure described in Ultradent Products Inc.'s U.S. Pat. No. 6,324,916 B1. The adhesive compositions of this invention comprise the so called one, two or three parts or bottles total etching, etch & rinse, or self etching system. In example 10, one part or bottle adhesive compositions are described. Examples 11 to 14 refer to self etching adhesive compositions comprising two parts or two bottles. Generally, Part A comprises solvents, hydrophilic monomers, initiator, and inhibitor with or without filler. Example 15 refers to Part B bonding compositions which, in general, comprise hydrophilic or hydrophobic dimethacrylate, initiator, and inhibitor. Compositions may or may not contain fillers.

### Example 2

In a three neck flask, under nitrogen atmosphere were mixed 10.00 g (0.0145 mol) of dimer acid diglycidyl ester, 2.54 g (0.0290 mol) of methacrylic acid, 0.05 g of 4-dimethoxyphenol (BHT) and 0.20 g (0.0016 mol) of 4-dimethylaminopyridine (DMAP). The reaction mixture was heated at 90° C. for 24 hours. Mid-IR indicated complete reaction with the disappearance of epoxy ring at approximately 960 cm⁻¹. To continue, 4.3695 g (0.0290 mol) of phatallic anhydride dissolved in 30 mL of tetrahydrofurane (THF) were added drop wise. Once addition was finished the reaction mixture was refluxed for 6 days. ¹H NMR indicated 75% pure material.

### Data for 1:

IR (KBr, cm⁻¹): ν 3052 (CH_{aromatic}), 2930, 2854 (CH_{aliphatic}), 1724, 1635 (CO), 1638 (═CH₂), 1600 (C═C)

¹H NMR (500 MHz, CDCl₃, ppm): δ 12.09 (s-br, COOH), 8.24, 8.20, 7.71, 7.67 (m, CH_{aromatic}), 6.15, 5.59 (m, ═CH₂), 5.15 (t, CH_{minority isomer}), 4.63-4.45 (m, OCH and OCH₂), 2.25 (m, CH₂), 1.98 (m, CH₃), 1.8-1.0 (m, CH and CH₂), 0.88 (m, CH₃).

¹³C{¹H} NMR (125 MHz, CDCl₃, ppm): b 174.0, 169.3, 167.2, 165.9 (CO), 134.0, 133.0, 132.5, 130.0, 128.6 (CH_{aromatic}), 136.0 (CH═CH₂), 126.2 (CH═CH₂), 72.2, 68.0, 65.2, 62.9, 61.3 (OCH₂ and OCH), from 40 to 20 (CH and CH₂ aliphatic), 17.9 (CH₃), 14.1 (CH₃).

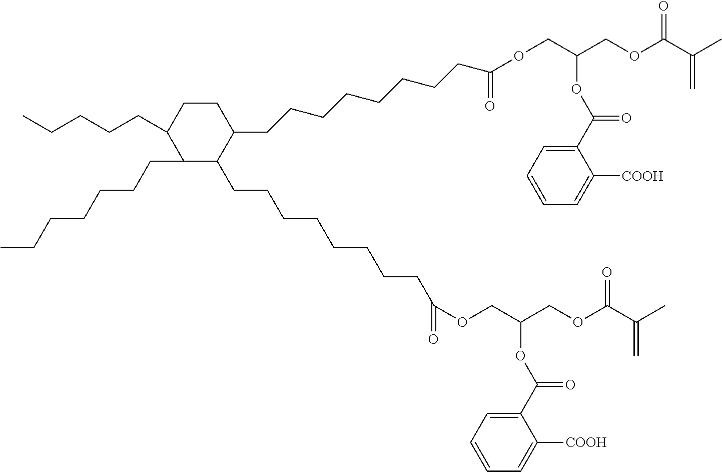 [SMILES: C=C(C)C(=O)OCC(COC(=O)CCCCCCCCC1CCC(CCCCC)C(CCCCCCC)C1CCCCCCCCC(=O)OCC(COC(=O)C(=C)C)OC(=O)c1ccccc1C(=O)O)OC(=O)c1ccccc1C(=O)O]

### Example 3

Under nitrogen atmosphere were mixed together 10.00 g (0.0145 mol) of dimer acid diglycidyl ester, 2.54 g (0.0290 mol) of methacrylic acid, 0.05 g of 4-dimethoxyphenol (BHT) and 0.20 g (0.0016 mol) of 4-dimethylaminopyridine (DMAP). The reaction mixture was heated at 90° C. for 24 hours. After allowing reaction mixture to reach room temperature, 2.95 g (0.0295 mol) of succinic anhydride dissolved in 30 mL of tetrahydrofurane (THF) were added drop wise. Once addition was finished the reaction mixture was refluxed for 4 days. ¹H NMR indicated 78% pure material.

### Data for 2:

IR (KBr, cm⁻¹): ν 2945, 2852 (CH_{aliphatic}), 1722, 1635 (CO), 1638 (═CH₂).

¹H NMR (500 MHz, CDCl₃, ppm): δ 11.89 (s-br, COOH), 6.11, 5.58 (m, ═CH₂), 4.5-4.2 (m, OCH and OCH₂), 2.25 (m, CH₂), 1.98 (m, CH₃), 1.8-1.0 (m, CH and CH₂), 0.88 (m, CH₃).

¹³C{¹H} NMR (125 MHz, CDCl₃, ppm): δ 177.3, 173.1, 167.2 (CO), 136.0 (CH═CH₂), 125.2 (CH═CH₂), 69.1, 64.5, 64.1 (OCH₂ and OCH), from 35 to 20 (CH and CH₂ aliphatic), 17.9 (CH₃), 14.1 (CH₃).

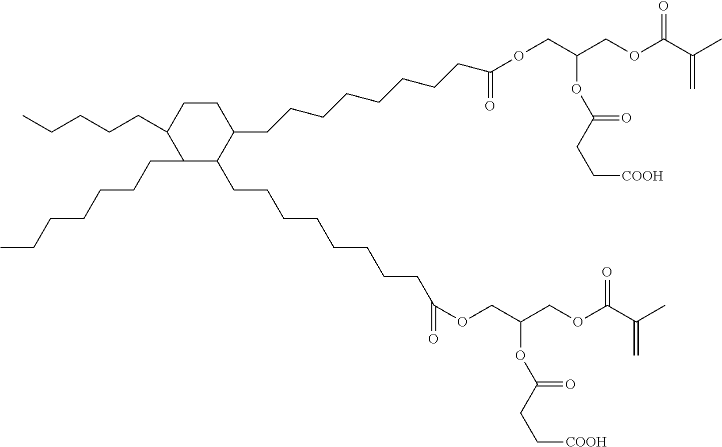 [SMILES: C=C(C)C(=O)OCC(COC(=O)CCCCCCCCC1CCC(CCCCC)C(CCCCCCC)C1CCCCCCCCC(=O)OCC(COC(=O)C(=C)C)OC(=O)CCC(=O)O)OC(=O)CCC(=O)O]

### Example 4

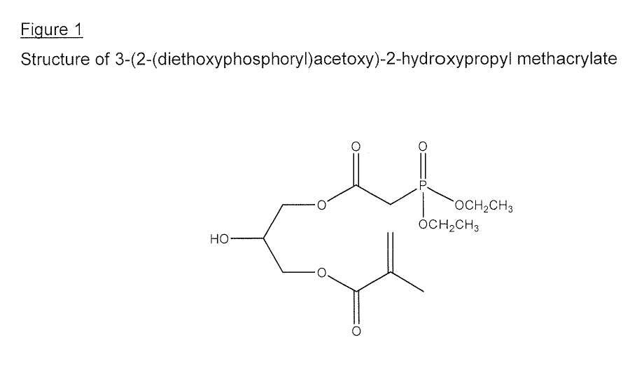

The synthesis of 3-(2-(diethoxyphosphoryl)acetoxy)-2-hydroxypropyl methacrylate (FIG. 1) was carried out by the reaction of glycidyl methacrylate (20 g, 0.1407 mol) with diethylphosphonic acid) (28.97 g, 0.1477 mol) in the presence of a catalytic amount of triethylamine in absence of solvent. ¹H NMR was used to follow the reaction. The procedure had been described previously on “Journal of Polymer Science: Part A: Polymer Chemistry,” Vol. 46, No 6 (2008): pp. 2290-2299.

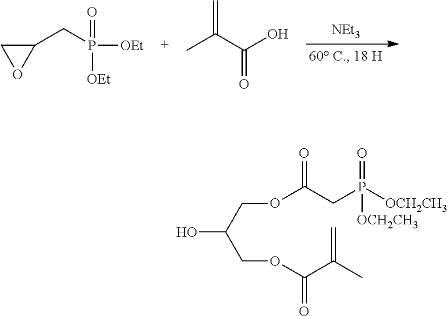

Two alternative methods to synthesize the same compound were described by Youssef et al, “Macromol. Chem. Phys”. 2003, 204, 1842-1850. The first method consists of the reaction of the phosphonate epoxide and methacrylic acid in the presence of tetraethyl ammonium bromide using anhydrous toluene as solvent. The second one is a esterification between diethyl dihydroxy-1,2-propyl phosphate and methacryloyl chloride.

### Example 5

The synthesis of compound 5 required two steps. In Step 1: 6.94 g (0.0205 mol) of material described in Example 3 were mixed with 5.89 g (0.01 mol) of dimer acid diisocyanate and two drops of dibutyl tin dilaurate as the catalyst. Reaction mixture was stirred at 40° C. for 18 hours. Mid-IR showed completion of reaction indicated by disappearance of isocyanate peak at 2271 cm⁻¹. Step 2: the product obtained in step 2 was dissolved in 15 mL of methylene chloride (CH₂Cl₂) and 2.51 g (0.0138 mol) of trimethylsilylbromide were added. Reaction mixture was reflux for 2 hours, solvent was removed with vacuum and then 20 mL of methanol were added. Reaction mixture was stirred at room temperature until next day. A slightly brown viscous liquid was obtained after evaporation of solvent. 4.26 g were obtained (yield: 93%).

### Data for 3:

IR (KBr, cm⁻¹): ν 3349 (COOH), 2925, 2854 (CH_{aliphatic}), 2304 (PO—H), 1725 (CO), 1638 (═CH₂), 1257 (P═O).

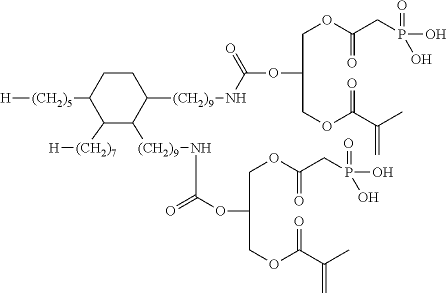

### Example 6

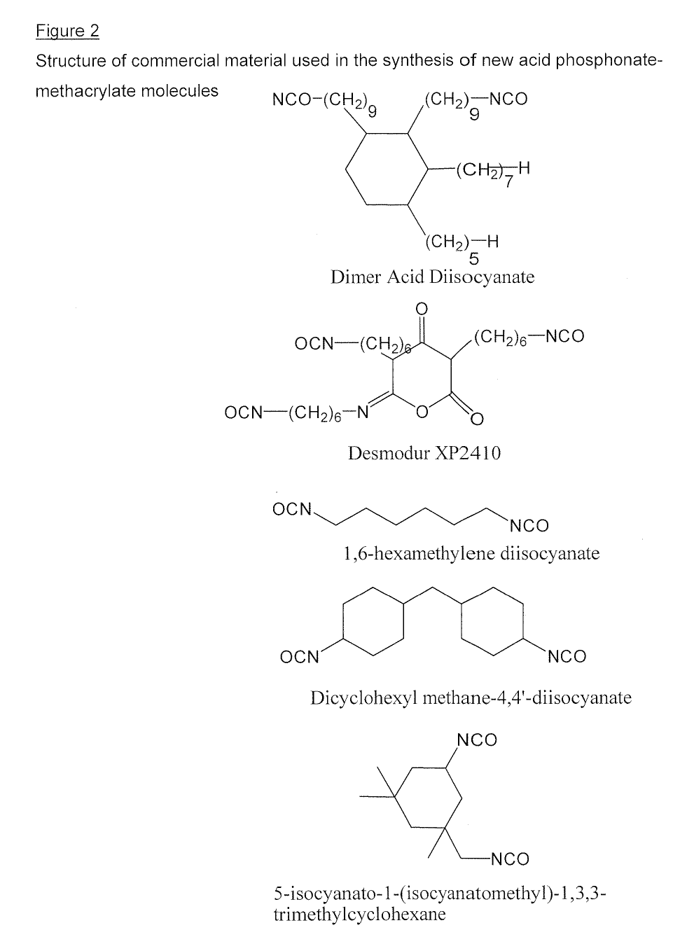

The synthesis of compound 6 required three steps. Step 1: 5.2446 g (0.0155 mol) of material described in Example 3 were mixed with 2.5261 g (0.005 mol) of Desmodur XP2410 (FIG. 2) and two drops of dibutyl tin dilaurate as the catalyst. Reaction mixture was stirred at 40° C. for 18 hours. Mid-IR showed completion of reaction indicated by disappearance of isocyanate peak at 2272 cm⁻¹. Step 2: 5.00 g (0.0033 mol) of product obtained in step 2 was dissolved in 15 mL of methylene chloride (CH₂Cl₂) and 2.12 g (0.0138 mol) (4.2 equivalents) of trimethylsilylbromide were added. Reaction mixture was reflux for 2 hours, solvent was removed with vacuum and then 20 mL of methanol were added. Reaction mixture was stirred at room temperature until next day. A slightly yellow viscous liquid was obtained after evaporation of solvent.

### Data for 4:

IR (KBr, cm⁻¹): ν 3360 (COOH), 2936, 2861 (CH_{aliphatic}), 2304 (PO—H), 1723, 1688 (CO), 1638 (═CH₂), 1247 (P═O).

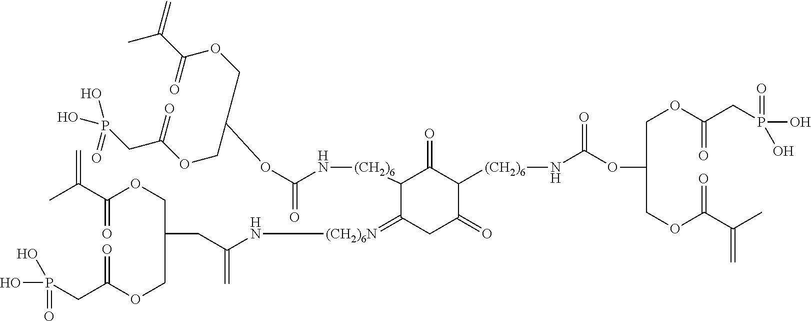

### Example 7

The synthesis of compound 7 required two steps. Step 1: In a round bottom flask were mixed 3.18 g (0.0189 mol) of 1,6-hexanediisocyanate and 13.45 g (0.0976 mol) of product synthesized in Example 3. To continue three drops of dibutyl tin dilaurate were added. Reaction mixture was stirred at 40° C. for 18 hours. Mid-IR showed completion of reaction indicated by disappearance of isocyanate peak at 2270 cm⁻¹. Step 2: Product obtained above was dissolved in 25 mL of methylene chloride (CH₂Cl₂) and 10.3 mL (4.2 equivalents) of trimethylsilylbromide were added. The resulting orange solution was reflux for 2 hours, and then solvent was evaporated. To continue, 20 mL of methanol were added and the solution was stirred at room temperature until next day. A yellow viscous liquid was obtained after evaporation of solvent.

### Example 8

The synthesis of compound 8 required two steps. Step 1: In a round bottom flask were mixed 15.52 g (0.1 mol) of 2-isocyantoethyl methacrylate and 34.19 g (0.101 mol) of product synthesized in Example 3 (FIG. 1), to continue three drops of dibutyl tin dilaurate were added. Reaction mixture was stirred at 40° C. for 18 hours. Mid-IR showed completion of reaction indicated by disappearance of isocyanate peak at 2270 cm″Step 2: Product obtained above was dissolved in 50 mL of methylene chloride (CH₂Cl₂) and 28.5 mL of trimethylsilylbromide were added. Reaction mixture was reflux for 2 hours, and then methylene chloride was removed with vacuum. To continue, 20 mL of methanol were added and the solution was stirred at room temperature until next day. A slightly yellow viscous liquid was obtained after evaporation of solvent.

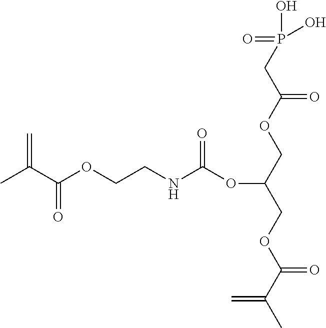 [SMILES: C=C(C)C(=O)OCCNC(=O)OC(COC(=O)CP(=O)(O)O)COC(=O)C(=C)C]

### Example 9

The following chart shows example of pH of 37% solution of new synthesized monomers compared with commercial monomers used in dental adhesives

| | Mol. Wt | |
|---|---|---|
| Monomer | (g/mol) | pH |
| 1 | 1145 | 3.7 |
| 2 | 1049 | 3.6 |
| PMGDMA | 675 | 3.7 |
| 4-META | 288 | 1.2 |

PMGDMA and 4-META structures described on Figure 2

### Example 10

The following chart shows example of viscosity, degree of conversion and volume shrinkage obtained for some of the synthesized monomer

| | | | | Volume |
|---|---|---|---|---|
| | Mol. Wt | Viscosity | Conversion | Shrinkage |
| Monomer | (g/mol) | (Pa*s) | (%) | (%) |
| 3 | 1151 | 1.65 ± 0.00 | 75.2 ± 0.6 | 2.6 ± 0.3 |
| 4 | 1349 | 15.5 ± 0.3 | 81 ± 1 | 2.8 ± 0.4 |
| 5 | 732 | 11.5 ± 0.1 | 82 ± 1 | 3.7 ± 0.2 |
| 6 | 437 | 11.9 ± 0.1 | 71.2 ± 0.4 | 5.8 ± 0.7 |

### Example 11

The following charts show example of one bottle self-etching compositions, with the amount of the components in wt % and pH obtained for each formulation

| Composition (%) | Fl | F2 | F3 | F4 |
|---|---|---|---|---|
| HEMA | 25 | 23 | 22 | 20 |
| (3) | 25 | 25 | 25 | 25 |
| Bis-GMA | 40 | 40 | 40 | 40 |
| Ethanol | 10 | 10 | 10 | 10 |
| Water | 0 | 2 | 3 | 5 |
| Total (%) | 100 | 100 | 100 | 100 |
| pH | a | 1.78 | 1.60 | 1.39 |

^{a} In anhydrous system pH cannot be determined

| Composition (%) | G1 | G2 | G3 | G4 |
|---|---|---|---|---|
| HEMA | 25 | 23 | 22 | 20 |
| (4) | 25 | 25 | 25 | 25 |
| Bis-GMA | 40 | 40 | 40 | 40 |
| Ethanol | 10 | 10 | 10 | 10 |
| Water | 0 | 2 | 3 | 5 |
| Total (%) | 100 | 100 | 100 | 100 |
| pH | a | 0.49 | 0.52 | 0.51 |

^{a} In anhydrous system pH cannot be determined

### Example 12

The following chart shows example of Part A for two bottle self-etching compositions, with the amount of the components in wt % and camphorquinone/amine as the photoinitiator system.

| | | Monomer Ratio | Ethanol/Water |
|---|---|---|---|
| Formulation No. | Monomers | Wt % | wt % |
| Part A (01) | HEMA/(3)/BisGMA | 20/25/35 | 18/2 |
| Part A (02) | HEMA/(4)/BisGMA | 20/25/36 | 18/2 |
| Part A (03) | HEMA/(5)/BisGMA | 20/25/37 | 17.5/2.5 |
| Part A (04) | HEMA/(6)/BisGMA | 20/25/38 | 18/2 |
| Part A (05) | HEMA/(3)/BisGMA | 15/25/30 | 28/2 |
| Part A (06) | HEMA/(4)/BisGMA | 15/25/31 | 28/2 |
| Part A (07) | HEMA/(5)/BisGMA | 15/25/32 | 27.5/2.5 |
| Part A (08) | HEMA/(6)/BisGMA | 15/25/33 | 28/2 |

Photoinitiator system: Camphorquinone 0.2 wt %/amine 0.8 wt %

### Example 13

The following chart shows example of Part A for two bottle self-etching compositions, with the amount of the components in wt % and Irgacure 1-819 as the photoinitiator system.

| | | Monomer Ratio | Ethanol/Water |
|---|---|---|---|
| Formulation No. | Monomers | Wt % | wt % |
| Part A (09) | HEMA/(3)/BisGMA | 20/25/35 | 18/2 |
| Part A (10) | HEMA/(4)/BisGMA | 20/25/36 | 18/2 |
| Part A (11) | HEMA/(5)/BisGMA | 20/25/37 | 17.5/2.5 |
| Part A (12) | HEMA/(6)/BisGMA | 20/25/38 | 18/2 |
| Part A (13) | HEMA/(3)/BisGMA | 15/25/30 | 28/2 |
| Part A (14) | HEMA/(4)/BisGMA | 15/25/31 | 28/2 |
| Part A (15) | HEMA/(5)/BisGMA | 15/25/32 | 27.5/2.5 |
| Part A (16) | HEMA/(6)/BisGMA | 15/25/33 | 28/2 |

Photoinitiator system: Irgacure I-819

### Example 14

The following chart shows example of Part A formulation for two bottle self-etching compositions, with the amount of the components in wt % with conversion and viscosity values.

| Formulation | | Monomer | Ethanol/ | Conversion | Viscosity |
|---|---|---|---|---|---|
| No. | Monomers | Ratio | Water | Day 0 | (mPa * s) |
| Part A (01) | HEMA/(3)/ | 20/25/35 | 18/2 | 100 ± 0 | 93.8 |
| | BisGMA | | | | |
| Part A (02) | HEMA/(4)/ | 20/25/36 | 18/2 | 98.0 ± 0.7 | 72.2 |
| | BisGMA | | | | |
| Part A (03) | HEMA/(5)/ | 20/25/37 | 17.5/2.5 | 99.0 ± 0.1 | 105.5 |
| | BisGMA | | | | |
| Part A (04) | HEMA/(6)/ | 20/25/38 | 18/2 | 99.0 ± 0.1 | 60.3 |
| | BisGMA | | | | |
| Part A (05) | HEMA/(3)/ | 15/25/30 | 28/2 | 97 ± 0.39 | 56 |
| | BisGMA | | | | |
| Part A (06) | HEMA/(4)/ | 15/25/31 | 28/2 | 97 ± 1 | 38.5 |
| | BisGMA | | | | |
| Part A (07) | HEMA/(5)/ | 15/25/32 | 27.5/2.5 | 98 ± 1 | 74.7 |
| | BisGMA | | | | |
| Part A (08) | HEMA/(6)/ | 15/25/33 | 28/2 | 98 ± 1 | 30.5 |
| | BisGMA | | | | |
| Part A (09) | HEMA/(3)/ | 20/25/35 | 18/2 | 100 ± 0.06 | 83 |
| | BisGMA | | | | |
| Part A (10) | HEMA/(4)/ | 20/25/36 | 18/2 | 100 ± 0.06 | 110 |
| | BisGMA | | | | |
| Part A (11) | HEMA/(5)/ | 20/25/37 | 17.5/2.5 | 98.0 ± 0.6 | 66.5 |
| | BisGMA | | | | |
| Part A (12) | HEMA/(6)/ | 20/25/38 | 18/2 | 100.0 ± 0.2 | 70.7 |
| | BisGMA | | | | |
| Part A (13) | HEMA/(3)/ | 15/25/30 | 28/2 | 100 ± 0.17 | 30 |
| | BisGMA | | | | |
| Part A (14) | HEMA/(4)/ | 15/25/31 | 28/2 | 100 ± 0.02 | 90.5 |
| | BisGMA | | | | |
| Part A (15) | HEMA/(5)/ | 15/25/32 | 27.5/2.5 | 100 ± 0 | 34 |
| | BisGMA | | | | |
| Part A (16) | HEMA/(6)/ | 15/25/33 | 28/2 | 100 ± 0 | 69.8 |
| | BisGMA | | | | |

### Example 15

The following chart shows example of Part A compositions shelf life studies at 5° C. Conversion of compositions was evaluated as function of time.

| Formulation | Day 0 | Day 15 | Day 30 | Day 45 | Day 60 | Day 90 |
|---|---|---|---|---|---|---|
| No. | Conversion | Conversion | Conversion | Conversion | Conversion | Conversion |
| Part A (01) | 100 ± 0 | 100 ± 0.4 | 97 ± 0.4 | 98 ± 0.98 | 99 ± 0.77 | 100 ± 0.18 |
| Part A (02) | 98.0 ± 0.7 | 99.0 ± .01 | 98 ± 0.56 | 96 ± 1.67 | 96 ± 2 | 97 ± 0.77 |
| Part A (03) | 99.0 ± 0.1 | 99.0 ± 0.3 | 99 ± 0.88 | 99 ± 0.57 | 96 ± 2 | 98 ± 0.70 |
| Part A (04) | 99.0 ± 0.1 | 99.00 ± 0.02 | 99 ± 0.59 | 99 ± 0.04 | 97 ± 0.6 | 99 ± 0.38 |
| Part A (05) | 97 ± 0.4 | 100 ± 0.4 | 73 ± 3 | 96 ± 0.48 | 98 ± 2.28 | 100 ± 0.45 |
| Part A (06) | 97 ± 1 | 91 ± 3 | 97 ± 0.37 | 97 ± 0.52 | 92 ± 2 | 97 ± 1.52 |
| Part A (07) | 98 ± 1 | 95 ± 4 | 93 ± 0.91 | 95 ± 3.8 | 94 ± 1 | 95 ± 2.26 |
| Part A (08) | 98 ± 1 | 98 ± 1 | 98 ± 0.34 | 99 ± 1.21 | 97 ± 0.2 | 99 ± 1.21 |
| WITH I-819 | | | | | | |
| Part A (09) | 100 ± 0.06 | 100 ± 0.57 | 100 ± 0 | 100 ± 0.02 | 100 ± 0.06 | 100 ± 0.07 |
| Part A (10) | 100 ± 0.06 | 100 ± .015 | 100 ± 0 | 100 ± 0.08 | 100 ± 0.06 | 100 ± 0.01 |
| Part A (11) | 98.0 ± 0.6 | 100 ± 0 | 100 ± 0.06 | 100 ± 0.03 | 100 ± 0.34 | 100 ± 0.01 |
| Part A (12) | 100.0 ± 0.2 | 100 ± 0 | 100 ± 0.21 | 100 ± 0.01 | 100 ± 0.2 | 100 ± 0.04 |
| Part A (13) | 100 ± 0.17 | 99 ± 0.73 | 100 ± 0 | 100 ± 0.03 | 99 ± 0.41 | 100 ± 0.13 |
| Part A (14) | 100 ± 0.02 | 100 ± 0.05 | 99 ± 0 | 100 ± 0.05 | 99 ± 0.33 | |
| Part A (15) | 100 ± 0 | 100 ± 0.35 | 100 ± 0.19 | 100 ± 0.03 | 99 ± 0.45 | 100 ± 0.02 |
| Part A (16) | 100 ± 0 | 100 ± 0.14 | 99 ± 1.10 | 100 ± 0.21 | 99 ± 0.58 | 100 ± 0.06 |

### Example 16

The following chart shows example of Part B formulations for two bottle self-etching compositions, with the amount of the components in wt % and camphorquinone/amine as the photoinitiator system.

| | | Monomer Ratio |
|---|---|---|
| Formulation No. | Monomers | Wt % |
| Part B (01) | BisGMA/TEGDMA | 70/30 |
| Part B (02) | BisGMA/HEMA | 70/30 |
| Part B (03) | BisEMA/DDCDMA | 70/30 |
| Part B (04) | BisEMA/DDCDMA/UDMA | 60/25/15 |
| Part B (05) | BisEMA/DDCDMA/(6)/UDMA | 60/12.5/12.5/15 |
| Part B (06) | BisEMA/DDCDMA/(5)/UDMA | 60/12.5/12.5/15 |
| Part B (07) | BisEMA/DDCDMA/(3)/UDMA | 60/12.5/12.5/15 |
| Part B (08) | BisEMA/DDCDMA/(4)/UDMA | 60/12.5/12.5/15 |

### Example 17

Shear bond strength (SBS) for two bottle self-etching experimental formulations and commercial products after 24 h at 37° C.

| | | Bond Strength | | | |
|---|---|---|---|---|---|
| | | (Mpa)/Dry | | | |
| Part A | Part B | Enamel | WetEnamel | DryDentin | WetDentin |
| 1 | 2 | 7.1 ± 2.1 | | | |
| 2 | 2 | 8.6 ± 1.9 | 8.9 ± 4.23 | 8.6 ± 3.91 | 14.5 ± 5.98 |
| 3 | 2 | 6.2 ± 1.7 | | | |
| 4 | 2 | 6.4 ± 2.1 | | | |
| 5 | 2 | 7.5 ± 2.7 | | | |
| 6 | 2 | 5.2 ± 1.0 | | | |
| 7 | 2 | 3.6 ± 1.6 | | | |
| 8 | 2 | 4.8 ± 1.9 | | | |
| 1 | 4 | 8.1 ± 6.2 | | | |
| 2 | 4 | 7.3 ± 1.8 | | | |
| 3 | 4 | 6.9 ± 1.3 | | | |

| Commercial Products | Bond Strength (MPa)/DryEnamel |
|---|---|
| Clearfil SE | 22.3 ± 3.15 |
| Pro-Options | 13.4 ± 2.47 |
| Confi-Quick | 9.9 ± 5.97 |
| Adper Prompt | 8.5 ± 1.24 |
| AdheSE | 9.3 ± 2.38 |
| Optibond FL | 19.6 ± 3.45 |
| Adper Scotchbond SE | 12.6 ± 1.31 |
| DC Etchbond S2 | 8.8 ± 1.95 |
| Ace All-bond SE | 9.5 ± 1.54 |

### Example 18

The present invention relates to process of producing the compound of formula (7), (8), (9), (10), and (11), comprising: protection of acid groups (ester formation), reaction of the alcohol with an acyl chloride (for example, methacryloyl chloride) in the presence of a base (for example, triethylamine), or reaction of the alcohol with an isocyanate (for example, isocyanatoethyl methacrylate) using a catalyst (for example Dibutyltin Dilaurate, or DBTDL), and cleavage of the protecting group.

1. A compound selected from the group consisting of:
(I) a compound of formula (I): 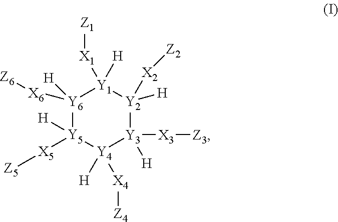
wherein the ring structure of formula (I) is saturated or contains up to three unsaturations, and wherein:
Y₁-Y₆, each independent from each other, is selected from the group consisting of: C, O, N, and S, with the proviso that at least three of Y₁-Y₆ are C, and wherein:
(i) when any one of Y₁-Y₆ is O, S, or an unsaturated nitrogen then the corresponding H, X₁-X₆ and Z₁-Z₆ are absent;
(ii) when any one of Y₁-Y₆ is a saturated nitrogen or an unsaturated carbon, then the corresponding H is absent;
X₁-X₆, each independent from each other, is a direct bond, or is selected from the group consisting of: ═O, ═S, ═N—, and R_{X}, wherein when any one of X₁-X₆ is ═O or ═S, then the corresponding Z₁-Z₆ is absent, wherein R_{X} is a C₁-C₁₅ group optionally having at least one unsaturation, branch and/or cycle, which is substituted up to 4 times or unsubstituted, and which may be interrupted by at least one O or S, wherein the substituents are each independently selected from the group consisting of —OH, —OR_{V}, ═O, ═S, —O₂CR_{V}, —SH, —SR_{V}, —SOCR_{V}, —NH₂, —NHR_{V}, —N(R_{V})₂, —NHCOR_{V}, —NRCOR_{V}, —I, —Br, —Cl, —F, —CN, —CO₂H, —CO₂R_{V}, —CHO, —COR_{V}, —CONH₂, —CONHR_{V}, —CON(R_{V})₂, —COSH, —COSR_{V}, —NO₂, —SO₃H, —SOR_{V}, and —SO₂R_{V}, wherein R_{V} is a linear, branched or cyclic alkyl of one to ten carbon atoms,
Z₁-Z₆, each independent from each other, is R_{S}, wherein R_{S} is selected from the group consisting of:
(a) H;
(b) a radical of formula (IV) 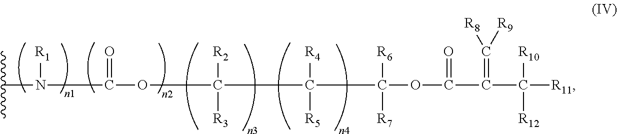
wherein:
n1 and n2, each independent from each other, is 0 or 1;
n3 and n4, each independent from each other, is 0 to 6;
R₁ to R₁₂, each independent from each other, is selected from the group consisting of:
(i) H,
(ii) R_{Y}, wherein R_{Y} is a C₁-C₆ group optionally having at least one unsaturation, branch and/or cycle, which is substituted up to 2 times or unsubstituted, and which may be interrupted by at least one O or S, wherein the substituents are each independently selected from the group consisting of —OH, —OR, ═O, ═S, —O₂CR, —SH, —SR, —SOCR, —NH₂, —NHR, —N(R)₂, —NHCOR, —NRCOR, —I, —Br, —Cl, —F, —CN, —CO₂H, —CO₂R, —CHO, —COR, —CONH₂, —CONHR, —CON(R)₂, —COSH, —COSR, —NO₂, —SO₃H, —SOR, and —SO₂R, wherein R is a linear or branched alkyl of one to three carbon atoms, and
(iii) a radical of formula (V): 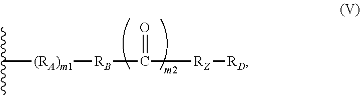
wherein:
R_{A} is R_{X}, as defined above, and m1 is 0 or 1,
R_{B} is O or S,
m2 is 0 or 1;
R_{Z} is selected from the group consisting of: 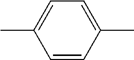
and a C₁-C₃ group optionally having at least one unsaturation or branch, which is substituted up to 2 times or unsubstituted, and which may be interrupted by at least one O or S, wherein the substituents are each independently selected from the group consisting of —OH, —OR, ═O, ═S, —O₂CR, —SH, —SR, —SOCR, —NH₂, —NHR, —N(R)₂, —NHCOR, —NRCOR, —I, —Br, —Cl, —F, —CN, —CO₂H, —CO₂R, —CHO, —COR, —CONH₂, —CONHR, —CON(R)₂, —COSH, —COSR, —NO₂, —SO₃H, —SOR, and —SO₂R, wherein R is a linear or branched alkyl of one to three carbon atoms,
R_{D} is selected from the group consisting of:
(i) 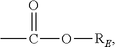
wherein R_{E} is H or R_{Y}, as defined above; and
(ii) 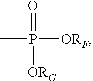
wherein R_{F} and R_{G}, each independent from each other, are selected from the group consisting of: H and R_{Y}, as defined above; and wherein at least one of R₁ to R₁₂ is a radical of formula (V); and
(II) a compound of formula (II): 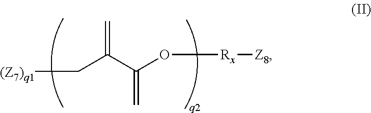
wherein:
Z₇ is R_{S}, as defined above,
q1 and q2, each independent from each other, is 0 or 1,
R_{X} is as defined above, and
Z₈ is a radical of formula (IV), as defined above; and
(III) a compound of formula (III): 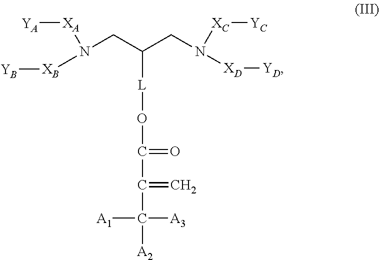
wherein:
X_{A}, X_{B}, X_{C}, and X_{D}, each independent from each other, is a direct bond or R_{Y}, wherein R_{Y} is C₁-C₆ group optionally having at least one unsaturation, branch and/or cycle, which is substituted up to 2 times or unsubstituted, and which may be interrupted by at least one O or S, wherein the substituents are each independently selected from the group consisting of —OH, —OR, ═O, ═S, —O₂CR, —SH, —SR, —SOCR, —NH₂, —NHR, —N(R)₂, —NHCOR, —NRCOR, —I, —Br, —Cl, —F, —CN, —CO₂H, —CO₂R, —CHO, —COR, —CONH₂, —CONHR, —CON(R)₂, —COSH, —COSR, —NO₂, —SO₃H, —SOR, and —SO₂R, wherein R is a linear or branched alkyl of one to three carbon atoms;
Y_{A}, Y_{B}, Y_{C}, and Y_{D}, each independent from each other, is H or COOH, with the proviso that at least two of Y_{A}, Y_{B}, Y_{C}, and Y_{D} are COOH;
L is selected from the group consisting of:
(a) a direct bond,
(b) 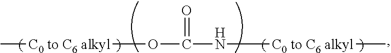
and
(c) R_{Y}, as defined above; and
A₁, A₂, and A₃, each independent of each other, are H or R_{Y}, as defined above.

2. (canceled)

3. (canceled)

4. The compound of claim 1, wherein in formula (I): (1) Y₁-Y₆ are all C; or (2) one of Y₁-Y₆ is O, and the remainder of Y₁-Y₆ are C; and wherein two of X₁-X₆ are ═O, and one of X₁-X₆ is ═N—.

5. (canceled)

6. The compound of claim 1, wherein in formula (I), at least 3 of X₁-X₆ are R_{X}, wherein R_{X} is a C₅-C₉ alkyl.

7. The compound of claim 1, wherein in formula (IV), R₁-R₁₂ are H, n3 and n4 are 1, and n2 is 1.

8.-10. (canceled)

11. The compound of claim 1, wherein in formula (IV), R₄ or R₆ is a radical of formula (V); and wherein in formula (V), R_{D} is 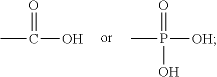
R_{Z} is a C₁-C₂ alkyl or 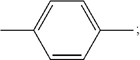
m2 is 1; R_{B} is O; R_{A} is a C₁ alkyl and m1 is 1.

12-15. (canceled)

16. The compound of claim 1, wherein in formula (III), X_{A}, X_{B}, X_{C}, and X_{D} are each C₁ unsubstituted alkyl groups; and wherein Y_{A} Y_{B}, Y_{C} and Y_{D} are each COOH.

17. (canceled)

18. The compound of claim 1, wherein in formula (III), L is selected from the group consisting of:
(a) 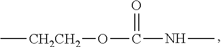
(b) 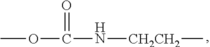
(c) 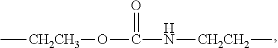
and
(d) a direct bond.

19. (canceled)

20. The compound of claim 1, wherein in formula (III), A₁, A₂, and A₃ are each H.

21. A compound selected from the group consisting of:

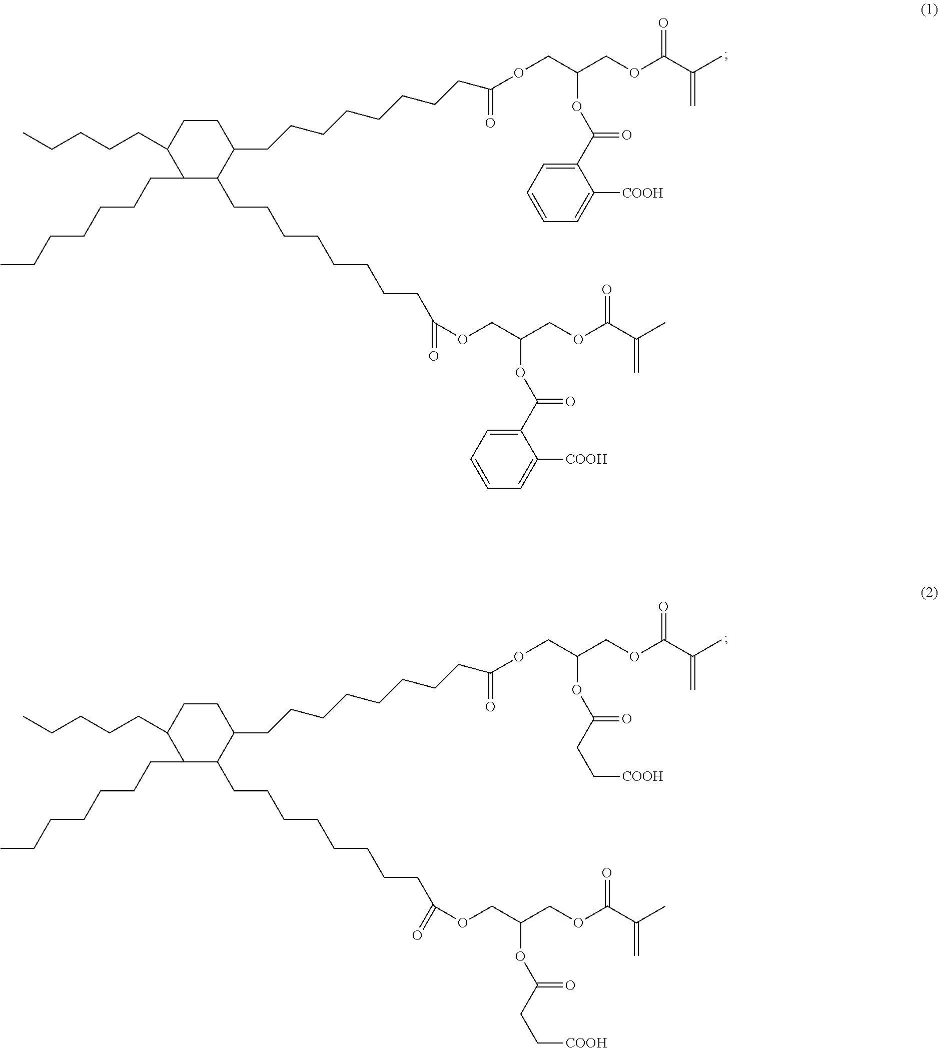

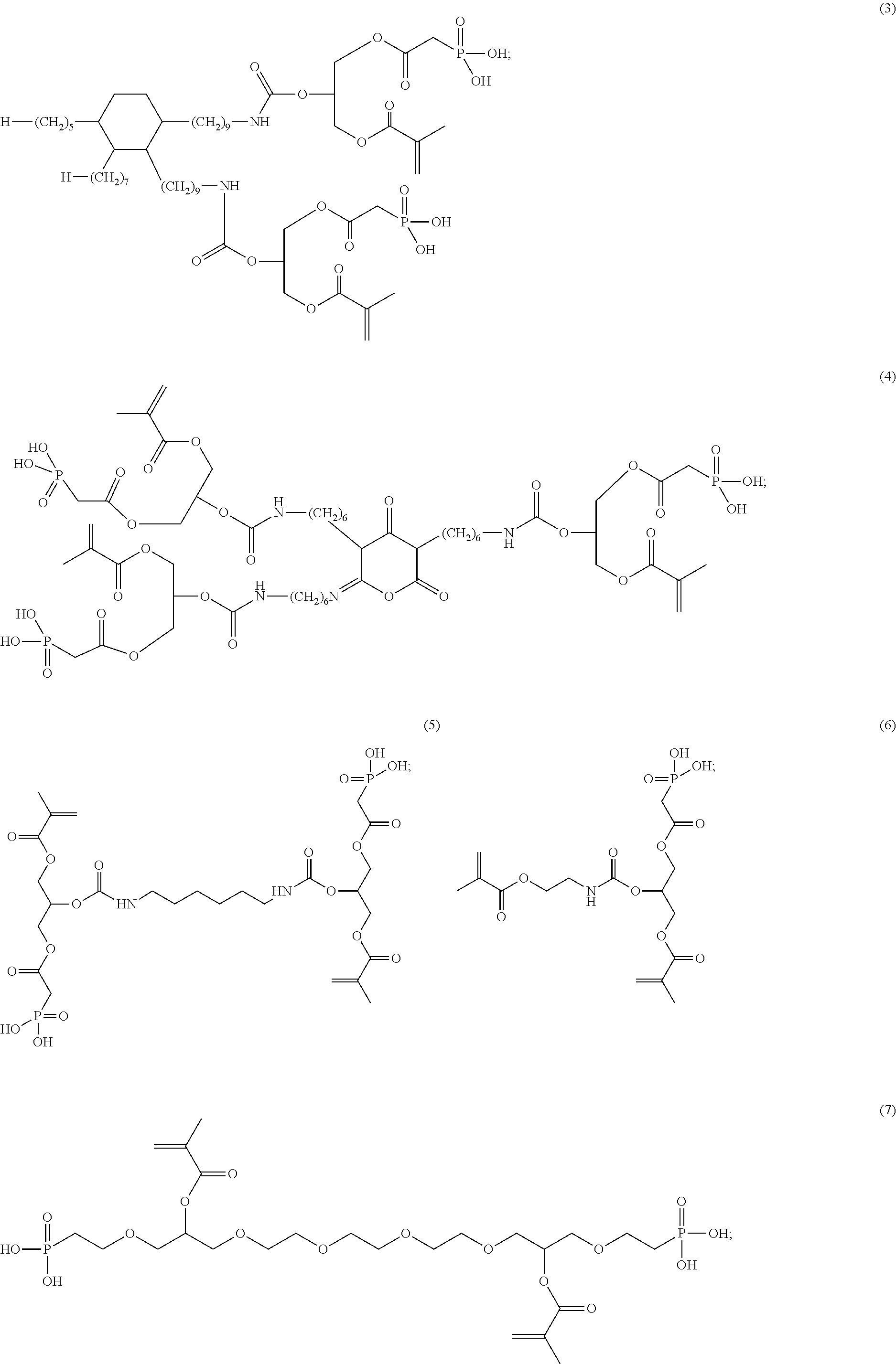

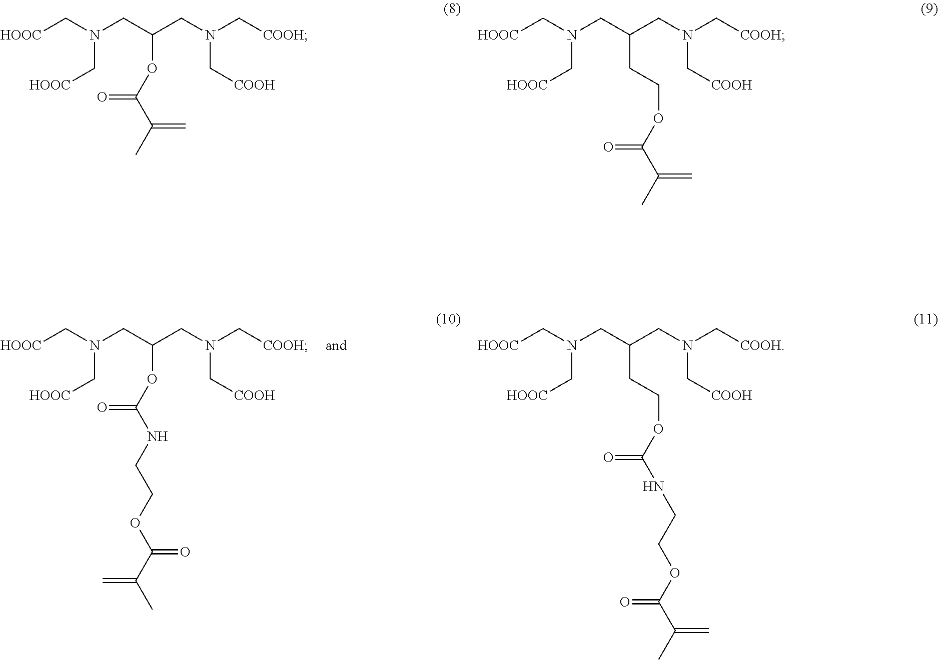 [SMILES: C=C(C)C(=O)OC(CN(CC(=O)O)CC(=O)O)CN(CC(=O)O)CC(=O)O ; C=C(C)C(=O)OCCC(CN(CC(=O)O)CC(=O)O)CN(CC(=O)O)CC(=O)O ; C=C(C)C(=O)OCCNC(=O)OC(CN(CC(=O)O)CC(=O)O)CN(CC(=O)O)CC(=O)O ; "and" ; C=C(C)C(=O)OCCNC(=O)OCCC(CN(CC(=O)O)CC(=O)O)CN(CC(=O)O)CC(=O)O]

22-31. (canceled)

32. A composition comprising the compound of formula (I), formula (II), or formula (III) of claim 1.

33. (canceled)

34. The composition of claim 32, wherein the composition further comprises a component selected from the group consisting of:
one or more polymerizable methacrylic monomers selected from the group consisting of: 2,2-bis[4-2(hydroxyl-3-methacryloyloxypropyl)phenyl]propane (Bis-GMA), dimer dicarbamate dimethacrylate (DDCDMA), 1,6-bis-[2-methacryloyloxyethoxycarbonylamino]-2,4,4-trimethylhexane (UDMA), and 2,2-bis(4-(2-Methacryloxyethoxy)phenylpropane (Bis-EMA), and Poly (ethylene glycol) dimethacrylate PEGDMA;
one or more hydrophilic methacrylates compounds selected from the group consisting of: 2-hydroxyethyl methacrylate (HEMA), triethylene glycol dimethacrylate (TEGDMA), ethylene glycoldimethacrylate (EGDMA), glycerol dimethcarylate (GDMA);
a water soluble organic solvent from the group of alcohol or ketones ethanol, propanol, acetone, methylethyl ketone and water; and
one or more filler materials selected from the group consisting of: silanized inorganic compounds, silica, silicate glass, quartz, barium silicate, strontium silicate, barium borosilicate, strontium borosilicate, borosilicate, alumina, zirconia, tin oxide, ytterbium fluoride, and pigments.

35-37. (canceled)

38. The composition of claim 32, wherein the composition further comprises one of more filler materials, wherein the particle sizes of the one or more filler materials are between about 0.001 to about 5.0 micrometers.

39. (canceled)

40. A dental application comprising the compound of formula (I), formula (II), or formula (III) of claim 1, wherein the dental application is selected from the group consisting of: dental adhesives; self adhesive restorative materials; permanent and temporary dental resin cements; light cure and chemical cure dental nanohybrid, microhybrid, and hybrid composites; dental nanohybrid and microhybrid flowable composites; temporary filling material; core build up material; and pit and fissure sealants.

41. (canceled)

42. A process of producing the compound of formula (1) of claim 21, comprising the steps of:
a) mixing diglycidyl ester, methacrylic acid, 4-dimethoxyphenol (BHT), and a base, wherein the base is preferably 4-dimethylaminopyridine (DMAP), triethylamine, or triphenyl phosphine,
b) adding phatallic anhydride dissolved in a polar solvent, wherein the solvent is preferably tetrahydrofuran.

43. A process of producing the compound of formula (2) of claim 21, comprising the steps of:
a) mixing diglycidyl ester, methacrylic acid, 4-dimethoxyphenol (BHT), and a base, wherein the base is preferably 4-dimethylaminopyridine (DMAP), triethylamine, or triphenyl phosphine,
b) adding succinic anhydride dissolved in a polar solvent, wherein the solvent is preferably tetrahydrofuran.

44. A process of producing the compound of formula (3) of claim 21, comprising the steps of:
a) mixing 3-(2-diethoxyphosphoryl)acetoxy)-2-hydroxypropyl methacrylate with diisocyanate, or mixing 3-(2-diethoxyphosphoryl)acetoxy)-2-hydroxypropyl methacrylate with a carboxylic acid and reacting with a dehydrating agent such as N,N′-dicyclohexylcarbodiimide (DCC)
b) adding a catalyst preferably selected from the group consisting of: dibutyl tin dilaurate, KKAT A209 (a zirconium chelate complex dissolved in a reactive diluent and t-butyl acetate), zirconium acetylacetonate, and dioctyltin dilaurate (DOTDL);
c) dissolving the resulting mixture in methylene chloride and trimethylsilylbromide;
d) removing solvent; and
e) adding methanol.

45. A process of producing the compound of formula (4) of claim 21, comprising the steps of:
a) mixing 3-(2-diethoxyphosphoryl)acetoxy)-2-hydroxypropyl methacrylate with Desmodur XP2410 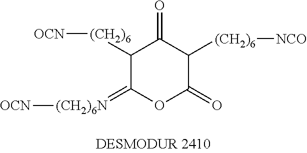
b) adding a catalyst preferably selected from the group consisting of: dibutyl tin dilaurate, KKAT A209 (a zirconium chelate complex dissolved in a reactive diluent and t-butyl acetate), zirconium acetylacetonate, and dioctyltin dilaurate (DOTDL);
c) dissolving the resulting mixture in methylene chloride and trimethylsilylbromide;
d) removing solvent; and
e) adding methanol.

46. A process of producing the compound of formula (5) of claim 21, comprising the steps of:
a) mixing 3-(2-diethoxyphosphoryl)acetoxy)-2-hydroxypropyl methacrylate with 1,6-hexanediisocyanate,
b) adding a catalyst preferably selected from the group consisting of: dibutyl tin dilaurate, KKAT A209 (a zirconium chelate complex dissolved in a reactive diluent and t-butyl acetate), zirconium acetylacetonate, and dioctyltin dilaurate (DOTDL);
c) dissolving the resulting mixture in methylene chloride and trimethylsilylbromide;
d) removing solvent; and
e) adding methanol.

47. A process of producing the compound of formula (6) of claim 21, comprising the steps of:
a) mixing 3-(2-diethoxyphosphoryl)acetoxy)-2-hydroxypropyl methacrylate with 2-isocyanoethyl methacrylate
b) adding a catalyst preferably selected from the group consisting of: dibutyl tin dilaurate, KKAT A209 (a zirconium chelate complex dissolved in a reactive diluent and t-butyl acetate), zirconium acetylacetonate, and dioctyltin dilaurate (DOTDL);
c) dissolving the resulting mixture in methylene chloride and trimethylsilylbromide;
d) removing solvent; and
e) adding methanol.

48. A process of producing the compound of formula (7), formula (8), formula (9), formula (10), and formula (11) of claim 21, comprising: protection of acid groups, reaction of the alcohol with an acyl chloride in the presence of a base, or reaction of the alcohol with an isocyanate using a catalyst, and cleavage of the protecting group.
